SEQUENCE LISTING (5 sequences)

SEQ ID NO: 1 - DNA, 584 bp, Zea mays
cgctgatcccaccggctgatagagttggcggccgggggagtgagtcagggatggcgtcggagccggtggcgcgggcggtggcggaggaggtgggccgctggggcagcatgaagcagacgggggtgaccctgcggtacatgatggagttcggctcccgccccacccagcgaaacctgctcctctccgcgcagttcctgcacaaggagctccccatccgcttcgcacgccgcgcgctcgagctcgactcgctgcccttcggcctctccaacaagcccgccatcctcaaggtgcgggactggtacttggactcattccgggacatcagatacttccctgaagtgaggagccggaacgacgagctcgctttcacgcagatgatcaatatggtcaaggtgcggcataacaatgtggttccaaccatggccttgggagtgcagcagctgaagaaggagctgggccgctcaaggaaggttccattcgaagtcgatgagatcgacgagttccttgaccggttctacatgtcaaggaatggcattcgcatgctgatagggcagcatgtggctttgcatgaccctaaaccggag

SEQ ID NO: 2 - DNA, 568 bp, Zea mays
gagctttcccgtgaggaaaatgtgtacatggcgaagctcgctgagcaggcggagaggtacgaggagatggttgagttcatggagaaggtagccaagactgttgactcggaggagctcactgtggaggagcgaaacctcttgtctgttgcatacaagaacgtcattggagcccgccgcgcctcatggcgcatcatctcctccatcgagcagaaggaggagggccgaggcaatgaggaccgagtaacactcatcaaggattatcgtggcaagattgagactgagctgaccaagatctgtgatggcatcctcaagctgcttgagacccatcttgtgccgtcttccactgcccccgagtccaaggtcttctatctcaagatgaagggtgattactacagataccttgctgagttcaagactggagctgagagaaaggacgccgctgagaacacgatggtggcatacaaggctgcccaagacattgctctggctgagcttgctcaacttaccttttaaggttggactggcacttaacttcttagtgtgctactatgagattctgaactaacct

SEQ ID NO: 3 - DNA, 404 bp, Zea mays
tatctactatactatactctaggaagcaaggacaccaccgccatggcagccaagatgcttgcattgttcgctctcctagctctttgtgcaagcgccactagtgcgacccatattccagggcacttgccaccagtcatgccattgggtaccatgaacccatgcatgcagtactgcatgatgcaacaggggcttgccagcttgatggcgtgtccgtccctgatgctgcagcaactgttggccttaccgcttcagacgatgccagtgatgatgccacagatgatgacgcctaacatgatgtcaccattgatgatgccgagcatgatgtcaccaatggtcttgccgagcatgatgtcgcaaatgatgatgccacaatgtcactgcgacgccgtctcgcagattatgct

SEQ ID NO: 4 - DNA, 349 bp, Zea mays
ctgttcacaaattatatgcaatcgcaagcgagcagaatggcgaggtccagtggtagtagaccagtggccctcgtgctgctggcgctgtgcgccgccgccctctcgtcggccacggtgaccgtgaatgagcccatcgccaatggcctctcctggagcttctacgacgtttcctgcccgtcggtggagggcatcgtgcgctggcacgtcgccgaggccctccgccgcgacatcggcatcgccgcggagctcatccgcatcttcttccacgactgcttcccgcatggctgcgacgcgtccgtcctcctgtctggttccatcagcgagcagatcgtagtacccaaccagacgc

SEQ ID NO: 5 - DNA, 505 bp, Zea mays
cccacgcgtcctcgcgactggcattattcatgtggaacaaatcactacaaagtgctgatggataagtttcacctcgtatccactgccttcctggagcttggtcaaggctatcaaaaggcaatcgaagaaatcactaggcgaatgggagcaggaatggcaaaatttatatgcaaggaggttgaaactgttgatgactatgacgagtattgtcactatgtagccgggctagttggttatggactttccaggctcttttatgctgctgggacggaagatctggctccagattcactatcaaattcaatgggtctctttttacagaaaactaatataattagggattatttggaggacataaatgagataccaaagtcccgcatgttctggcctcgagagatatggagtaaatatgcagataaactcgaggatttcaaatatgaggaaaataccgaaaaggcagtacaatgcttgaacgatatagtgacgaatgcactgattcatgctg

(19) United States
(12) Patent Application Publication
(10) Pub. No.: US 2004/0067514 A1
(43) Pub. Date: Apr. 8, 2004

(54) Methods for detecting translation initiation codons in nucleic acid sequences

(76) Inventor: Jack E. Tabaska, Creve Coeur, MO (US)

Correspondence Address: Pamela J. Sisson, Patent Department, E2NA, Monsanto Company, 800 N. Lindbergh Blvd., St. Louis, MO, 63167, US

(21) Appl. No.: 10/620,796

(22) Filed: Jul. 16, 2003

Related U.S. Application Data
(60) Provisional application No. 60/396,908, filed on Jul. 17, 2002.

(51) Int. Cl. C12Q 1/68; G06F 19/00; G01N 33/48; G01N 33/50

(52) U.S. Cl. 435/6; 702/20

(57) ABSTRACT

The present invention relates to a program to be used as a bioinformatics tool for analyzing files of nucleotide sequence data and finding the translation initiation codon. The invention described here uses Quadratic Discriminant Analysis (QDA) to determine the translation initiation codon in a nucleotide sequence. This program uses several components to provide a probability score for each potential translation initiation codon in a cDNA sequence. The components for each potential initiation codon in the sequence include a scoring method for (1) a model of the initiation consensus sequence, (2) the length of the upstream 5′ untranslated region, (3) the third base composition downstream from the +6 position, (4) a codon transition score, and (5) a bulk monomer composition transition.

### INCORPORATION OF SEQUENCE LISTING AND TABLES

Two paper copies of the sequence listing and a computer-readable form of the sequence listing on CD-ROM containing the file named “52529.ST25.txt”, which is 3,819 bytes (measured in MS-DOS) and was created on Jul. 14, 2003, are herein incorporated by reference.

Two copies of Table 4 (Table 4 Copy 1 and Table 4 Copy 2) all on CD-ROMs, each containing the file named “table4.txt”, which is 210,840 bytes (measured in MS-DOS) and was created on Jul. 12, 2002, are herein incorporated by reference.

### INCORPORATION OF COMPUTER PROGRAM LISTING APPENDIX

This application contains a first computer program-listing appendix, which is contained on two identical CD-ROMs, Copy 1 and Copy 2, labeled “52529_ComputerFiles” both of which are herein incorporated by reference. Both CD-ROMs each contain the following files:

1) “psue_sites.fa” which is 129,088 bytes in size and was created on Feb. 21, 2002;

2) “true_sites.fa” which is 9,383 bytes in size and was created on Feb. 21, 2002;

3) “mrna_no_hit.fa” which is 164,539 bytes in size and was created on Feb. 21, 2002;

4) “pseu2.fa” which is 328,298 bytes in size and was created on Feb. 21, 2002;

5) “true2.fa” which is 75,598 bytes in size and was created on Feb. 21, 2002;

6) “init.net” which is 4,244 bytes in size and was created on Feb. 21., 2002;

7) “init.qdf” which is 635 bytes in size and was created on Feb. 21, 2002;

8) “f_mono.score” which is 239 bytes in size and was created on Feb. 21, 2002;

9) “codon.odds” which is 3,700 bytes in size and was created on Feb. 21, 2002;

10) “monomer.score” which is 88 bytes in size and was created on Feb. 21, 2002;

11) “2mers.local.score” which is 665 bytes in size and was created on Feb. 21, 2002;

12) “3mers.local.score” which is 2,712 bytes in size and was created on Feb. 21, 2002;

13) “autocorr.dat” which is 380 bytes in size and was created on Feb. 21, 2002;

14) “auto3. dat” which is 433 bytes in size and was created on Feb. 21, 2002; and

15) “scale.dat” which is 7 bytes in size and was created on Feb. 21, 2002.

This application contains a second computer program-listing appendix, which is contained on two identical CD-ROMS, copy 1 and copy 2, labeled “52529_computerflles_source.tar.gz” both of which are herein incorporated by reference. Both CD-ROMS each contain the following files:

1) “bayes_net.cpp.ascii” which is 3,615 bytes in size and was created on Oct. 29, 2001;

2) “bayes_net.h.ascii” which is 2,186 bytes in size and was created on Oct. 29, 2001;

3) “bayesian.h.ascii” which is 784 bytes in size and was created on Oct. 29, 2001;

4) “defaults.h.ascii” which is 754 bytes in size and was created on Oct. 29, 2001;

5) “f-mono.cpp.ascii” which is 5,617 bytes in size and was created on Oct. 29, 2001;

6) “f_mono.h.ascii” which is 1,622 bytes in size and was created on Oct. 29, 2001;

7) “fscodon.cpp.asscii” which is 4,852 bytes in size and was created on Oct. 29, 2001;

8) “fscodon.h.ascii” which is 1,413 bytes in size and was created on Oct. 29, 2001;

9) “init_scan.cpp.ascii” which is 3,066 bytes in size and was created on Oct. 29, 2001;

10) “init_scan.h.ascii” which is 1378 bytes in size and was created on Oct. 29, 2001;

11) “lscale.cpp.ascii” which is 468 bytes in size and was created on Oct. 29, 2001;

12) “lscale.h.ascii” which is 934 bytes in size and was created on Oct. 29, 2001;

13) “machine.cpp.ascii” which is 1,205 bytes in size and was created on Oct. 29, 2001;

14) “machine.h.ascii” which is 679 bytes in size and was created on Oct. 29, 2001;

15) “main.cpp.ascii” which is 3,641 bytes in size and was created on Oct. 29, 2001;

16) “makefile.ascii” which is 1,661 bytes in size and was created on Oct. 29, 2001;

17) “monomer.cpp.ascii” which is 1,470 bytes in size and was created on Oct. 29, 2001;

18) “monomer.h.ascii” which is 826 bytes in size and was created on Oct. 29, 2001;

19) “my_except.h.ascii” which is 1,643 bytes in size and was created on Oct. 29, 2001;

20) “parameters.h.ascii” which is 2,638 bytes in size and was created on Oct. 29, 2001;

21) “qda.cpp.ascii” which is 2,425 bytes in size and was created on Oct. 29, 2001;

22) “qda.h.ascii” which is 1,419 bytes in size and was created on Oct. 29, 2001;

23) “sequence.cpp.ascii” which is 2,710 bytes in size and was created on Oct. 29, 2001;

24) “sequence.h.ascii” which is 14,608 bytes in size and was created on Oct. 29, 2001;

25) “vector.cpp.ascii” which is 2,797 bytes in size and was created on Oct. 29, 2001; and

26) “vector.h.ascii” which is 4,239 bytes in size and was created on Oct. 29, 2001.

This application contains a third computer program-listing appendix, which is contained on two identical CD-ROMS, copy 1 and copy 2, labeled “52529_computerfiles_xval3.tar.gz” both of which are herein incorporated by reference. Both CD-ROMS each contain the following files:

1) “all_est.pts.ascii” which is 4,975 bytes in size and was created on Jan. 10, 2002;

2) “datalist.cpp.ascii” which is 994 bytes in size and was created on Jan. 10, 2002;

3) “datalist.h.ascii” which is 355 bytes in size and was created on Jan. 10, 2002;

4) “datapoint.h.ascii” which is 506 bytes in size and was created on Jan. 10, 2002;

5) “final.var.est.ascii” which is 27 bytes in size and was created on Jan. 10, 2002;

6) “main.cpp.ascii” which is 1,455 bytes in size and was created on Jan. 10, 2002;

7) “makefile.ascii” which is 907 bytes in size and was created on Jan. 10, 2002;

8) “my_except.h.ascii” which is 958 bytes in size and was created on Jan. 10, 2002;

9) “performance.h.ascii” which is 1,166 bytes in size and was created on Jan. 10, 2002;

10) “qda.cpp.ascii” which is 2,531 bytes in size and was created on Jan. 10, 2002;

11) “qda.h.ascii” which is 1,220 bytes in size and was created on Jan. 10, 2002;

12) “qda_train.cpp.ascii” which is 8,275 bytes in size and was created on Jan. 10, 2002;

13) “qda_train.h.ascii” which is 1,154 bytes in size and was created on Jan. 10, 2002;

14) “set.cpp.ascii” which is 1,757 bytes in size and was created on Jan. 10, 2002;

15) “set.est.ascii” which is 78 bytes in size and was created on Jan. 10, 2002;

16) “set.h.ascii” which is 511 bytes in size and was created on Jan. 10, 2002;

17) “subgroup.h.ascii” which is 831 bytes in size and was created on Jan. 10, 2002;

18) “vector_math.h.ascii” which is 3,064 bytes in size and was created on Jan. 10, 2002;

19) “xval.cpp.ascii” which is 7,722 bytes in size and was created on Jan. 10, 2002; and

20) “xval.h.ascii” which is 333 bytes in size and was created on Jan. 10, 2002.

The present invention relates to the field of bioinformatics. More specifically the present invention relates to the detection and analysis of translation initiation codons in nucleic acid sequences.

Large-scale nucleic acid sequencing efforts are currently being carried out: with a variety of organisms in an effort to better understand and study their molecular processes. One of the challenges associated with this is finding new approaches to deal with the both the volume and the complexity of this data and producing analysis and computing tools in order to advance our understanding of the sequence information. Nucleic acid sequences themselves are not informative; sequences must be analyzed by comparative methods against existing databases to develop hypotheses concerning relationships and function. Without methods for analyzing and annotating nucleic acid sequence data there will soon be a large amount of data for which very little is known. Automated systems for sequence analysis are currently available from many sources, but one of the problems with gene prediction is still the large number of false positives and the accuracy of the translation initiation codon, intron, exon, and open reading frame determination.

It has been reported that the minimal translation initiation consensus sequence in eukaryotic mRNA may be commonly GCCACCATGG (M Kozak (1996) “Interpreting cDNA sequences: some insights from studies on translation.” Mammalian Genome 7: 563-574). This consensus sequence for the context of the translation initiation codon can be useful in identifying the possible translation initiation codon and correct open reading frame in new genes. The consensus sequence alone, however, is not necessarily indicative of the true translation initiation codon in a nucleic acid sequence. A problem faced by bioinformaticists and molecular biologists is to differentiate between the true translation initiation codon and internal methionine codons (also coded for by the ATG triplet). Sequencing errors leading to frameshifts in the sequence can cause additional complications. Many programs have been developed in the past decade for automated searching and analyzing of nucleic acid sequence data. These programs can be based on neural networks, hidden Markov models, or discriminant analysis.

Programs based on neural networks are available for gene prediction in nucleic acid sequence. A neural network is a method in which a computer is presented with huge amounts of data on a particular problem and programmed to pull out patterns. Neural networks are intended to model the human learning process by adapting to a training data set. Neural networks, however, may recognize biologically irrelevant features. GrailEXP, GeneParser2, and GeneBuilder are programs that use neural networks for analyzing nucleic acid sequences. GrailEXP is a suite of tools that can be used to locate protein-coding genes within nucleic acid sequence (Y Xu and E C Uberbacher (1997) “Automated Gene Identification in Large-Scale Genomic Sequences” Journal of Computational Biology Volume 4 Issue 3). GrailEXP can also be used to locate EST/mRNA alignments, certain types of promoters, polyadenylation sites, CpG islands, and repetitive elements. GeneParser2 is a program that can be used for the identification of protein coding regions in genomic nucleic acid sequences. (EE Snyder, G D Stormo (1995) “identification of Coding Regions in Genomic DNA.” Journal of Molecular Biology 248: 1-18). GeneBuilder is another tool tha can be used for prediction and analysis of protein-coding gene structures in genomic nucleic acid sequences (L Milanesi, D D'Angelo, I B Rogozin (1999) “GeneBuilder: interactive in silico prediction of genes structure.” Bioinformatics 15(7): 612-621).

Hidden Markov models (HMM) are a modeling technique that may be used to develop programs for sequence analysis. A hidden Markov model is a general statistical modeling technique for linear problems such as nucleic acid sequences that: describes a probability distribution over a potentially infinite set of sequences (S R Eddy (1996) “Hidden Markov Models.” Current Opinion in Structural Biology 6: 361-365). It can be difficult, however, to incorporate potentially useful information (such as ATG position) into an HMM because they cannot model nonlinear or irregular correlations. GENSCAN, GeneMark, and ESTScan are programs that use hidden Markov models for analyzing nucleic acid sequences. The GENSCAN program can be used for predicting the locations and exon-intron structures of genes in genomic nucleic acid sequences from a variety of organisms. (C Burge and S Karlin (1997) “Prediction of complete gene structures in human genomic DNA.” Journal of Molecular Biology 268:78-94). GeneMark is a family of gene prediction programs that can be used for finding gene locations within unannotated genomic nucleic acid sequence (M Borodovsky and J McIninch (1993) “GeneMark: parallel gene recognition for both DNA strands.” Computers & Chemistry 17(19): 123-1 33). ESTScan is a program that can be used to detect coding regions in EST sequences (C Iseli, C V Jongeneel, and P Bucher (1999) “ESTScan: A program for detecting, evaluating, and reconstructing potential coding regions in EST sequences.” Intelligent Systems for Molecular Biology Proceedings 7: 138-148).

Another modeling technique is Quadratic Discriminant Analysis (QDA). Programs use Quadratic Discriminant Analysis for analyzing nucleic acid sequences. There exists a program that can be used to predict internal coding exons in genomic nucleic acid sequences (M Q Zhang (1997) “Identification of protein coding regions in the human genome by Quadratic Discriminant Analysis.” Proceedings of the National Academy of Science USA 94: 565-568). Other programs exist that can be used for predicting polyadenylation signals in genomic nucleic acid sequences (J E Tabaska and M Q Zhang (1999) “Detection of polyadenylation signals in human DNA sequences.” Gene 231: 77-86) or for predicting 3′ terminal exons in genomic nucleic acid sequences (J E Tabaska, R V Davuluri, and M Q Zhang (2001) “identifying the 3′-terminal exon in human DNA.” Bioinformatics 17(7): 602-607).

Bayes networks are complex diagrams that can be used in Quadratic Discriminant Analysis based programs. Bayes networks organize the body of knowledge in any given area by mapping out cause-and-effect relationships among key variables and encoding them with numbers that represent the extent to which one variable is likely to affect another (B P Carlin and T A Louis (2000) Bayes and Empirical Bayes Methods for Data Analysis. Chapman & Hall/CRC, Boca Raton). Programmed into computers, these systems can automatically generate optimal predictions or decisions even when key pieces of information are missing. Bayes networks offer an efficient way to deal with the lack or ambiguity of information that has hampered previous systems.

What is currently needed in the art is a program capable of predicting translation initiation codons in nucleic acid sequences using a probabilistic method for compensating for frameshift-inducing sequencing errors (insertion or deletion of a nucleotide) and having an adjustable length scaling parameter that would allow for use on nucleic acid sequences with full-length 5′ untranslated regions to determine the position of the translation initiation codon.

### BRIEF SUMMARY OF THE INVENTION

The present invention includes and provides a method to be used as a bioinformatics tool for analyzing files of nucleic acid sequence data to find translation initiation codons. In accomplishing the foregoing, there is provided, in accordance with one aspect of the present invention, a method for use in a computer system for finding translation initiation codons in a nucleotide sequence, comprising (1) analyzing a data set to measure a combination of features of initiator codons and pseudoinitiator codons and to produce a set of numerical values for said combination of features, (2) evaluating scoring functions by reading a sequence in the vicinity of an ATG triplet and using said scoring functions and said scoring function's parameters to return a numerical score that quantifies how much said ATG triplet resembles an initiator codon, (3) generating a quadratic discriminant function through selection of a combination of variables that optimally classifies ATG triplets in a nucleotide sequence as initiator codons or as pseudoinitiator codons based on the output of said scoring functions and through the use of Quadratic Discriminant Analysis, and (4) using said quadratic discriminant function to analyze a data set of nucleotide sequences by evaluating scoring functions for each ATG triplet in said sequences and to calculate the probability of an initiator codon at a position using the output of said analysis.

### DETAILED DESCRIPTION OF THE INVENTION

The following detailed description of the invention is provided to aid those skilled in the art in practicing the present invention. Even so, the following detailed description should not be construed to unduly limit the present invention as modifications and variations in the embodiments discussed herein may be made by those of ordinary skill in the art without departing from the spirit or scope of the present inventive discovery.

The present invention includes and provides a method for analyzing nucleic acid sequences. The method is a computer program using a Quadratic Discriminant Function (QDF) comprised of a combination of at least two variables to perform the task of finding translation initiation codons in a nucleic acid sequence. The program may use several components to provide a probability score for each potential translation initiation codon in a nucleic acid sequence.

As used herein, the terms “nucleic acid sequence”, “nucleotide sequence” and “DNA sequence” include a nucleic acid sequence of any nucleic acid as is generally understood in the art. The nucleic acid can be DNA, cDNA, genomic DNA, raw DNA, RNA, mRNA, expressed nucleic acid sequence tags (ESTs), or any other form of nucleic acid regardless of whether or not the nucleic acid actually codes for a protein. Nucleic acid sequences can be derived from any natural or artificial source, including prokaryotic and eukaryotic organisms. The nucleic acid sequence notation conventionally used (A=Adenine, C=Cytosine, G=Guanine, and T=Thymine) is used herein in addition to nucleic acid sequence notation indicating uncertainty with respect to the identification of one or more bases in a nucleic acid sequence, for example IUB nomenclature such as N=A, C, G, or T.

The terms “initiator codon”, “start codon”, and “translation initiation codon” as used herein refer to the ATG triplet where mRNA translation and protein sequence begins.

The terms “pseudoinitiator” and “pseudoinitiator codon” as used herein refer to an ATG triplet in a DNA sequence that does not serve as a translation initiation codon.

The present invention can use one or more training data sets. A training data set can be used by the present invention for measuring one or more features of initiator or pseudoinitiator codons in a nucleic acid sequence to produce a set of numerical values for said feature.

As used herein the terms “training data set”, “training sequence data set”, and “training data” refer to a collection of sequence information that can be used for functions such as performing statistical characterization or training of the quadratic discriminant function.

As used herein the terms “data set” and “sequence data set” refer to a collection of sequence information. More preferably the sequence information represents nucleic acid sequence information stored in a computer readable form.

As used herein, the terms “statistical training set” and “discriminant training set” refer to a training data set of nucleic acid sequences containing at least 50 sequences, each sequence of which contains known translation initiation codons and pseudoinitiator codons. A statistical training set refers to a training set used for statistical characterization. A discriminant training set refers to a training set used for training the quadratic discriminant function.

As used herein, the term “true training set” refers to a data set of nucleic acid sequences comprised of known initiator codons and their surrounding sequence context. A true training set can be used for discriminant training, statistical characterization, or both.

As used herein the term “psuedo training set” refers to a data set of nucleic acid sequences comprised of pseudoinitiators and their surrounding sequence context. A psuedo training set can be used for discriminant training, statistical characterization, or both.

The tern “cDNA” as used herein refers to any double-stranded DNA molecule that is complementary to and derived from any RNA molecule. The term cDNA typically refers to a double-stranded molecule that is complimentary to and derived from any mRNA molecule although the cDNA may also be complimentary to and derived from tRNA, snRNA, rRNA or hnRNA sequences. By “cDNA sequence” it is meant a full or partial DNA sequence of a cDNA molecule. A cDNA sequence typically does not contain complete promoters, introns, or large non-coding regions of genomic DNA sequence. A cDNA sequence potentially contains untranslated regions at the 5′ and 3′ ends with the uninterrupted protein-coding or molecular-coding DNA sequence in between. By “cDNA insert” or “cDNA molecule” it is meant a cDNA molecule that, for the most part, is the complementary sequence of whole or a portion of an RNA molecule and is derived from an RNA molecule. A cDNA insert may optionally contain adapter sequences or other sequences used in generating the cDNA molecule using standard molecular biology techniques.

A “full-length cDNA sequence” as used herein refers to a cDNA sequence having the entire coding sequence of the corresponding RNA molecule. The sequence of a full-length cDNA molecule may be recognized as having a nucleotide alignment containing the annotated start and stop codon of a coding sequence.

The terms “coding sequence” and “protein-coding sequence” as used herein refer to the segment of DNA that directly codes for a protein product.

The terms “EST” and “EST sequence” as used herein refer to a full-length cDNA sequence or a portion of a full-length cDNA sequence, usually produced from a protein-coding region.

The terms “genomic”, “genomic sequence”, “genomic DNA”, “genomic nucleic acid sequence”, and “genomic DNA sequence” as used herein refer to nucleic acid sequence that may contain both coding and non-coding regions. Coding regions may include sequences useful for the generation of protein or RNA molecules. Examples of such sequences include, but are not limited to, protein coding sequence. Non-coding regions may include regulatory sequences comprised of sequences useful for regulating transcription, translation, stability, replication, length, molecular interactions, enhancement or suppression of expression, or location. Examples of such regulatory sequence include, but are not limited to, 3′ untranslated regions, 5′ untranslated regions, enhancers, introns, leader sequences, telomeres, and splice sites or other processing sites.

The present invention may use statistical characterization of one or more features with at least one training set to produce one or more parameters for one or more scoring functions.

As used herein, “statistical characterization” refers to a process comprised of measuring one or more features of initiator and pseudoinitiator codons and subsequently producing a set of numerical values. The set of numerical values produced from statistical characterization of data may then become parameters for the scoring functions as described herein. As used herein, the term “parameter” refers to a numerical measurement on a data set that characterizes one of its features.

As used herein, the term “feature” refers to any characteristic. In particular, feature refers to characteristics related to initiator or psuedo-initiator codons. In one embodiment, the features are selected from a list of features provided in Table 1. Each feature is described in detail below.

TABLE 1
Feature Name
Kozak Consensus
Frame-Specific Base Composition
Codon Usage
Bulk Monomer Composition
Bulk n-mer Composition
In-Frame Hexamer Composition
Diamino Acid Usage
Autocorrelation

Some of the statistics described herein are initially computed in the form of the following equation (1):

$$y = ln ( \frac{{p}_{pseudo}}{{p}_{true}} ) \qquad ( 1 )$$

known as log odds ratios. (LOR) may be used to simplify probability calculations and are well known to one skilled in the art. In the scoring functions described herein, log odds ratios converted to Bayesian probabilities (p) using the following equation (2):

$$p = \frac{1}{1 + {ⅇ}^{LOR + LOP}} \qquad ( 2 )$$

where LOP is the log odds of the Bayesian prior π. LOP is calculated using the following equation (3):

$$LOP = ln ( \frac{1 - π}{π} ) \qquad ( 3 )$$

Each analysis described below may be used with a true training set, comprised of known initiator codons and their surrounding sequence context, and a pseudo training set, comprised of pseudoinitiators and their surrounding sequence context.

The Kozak consensus may be modeled using a Bayes network (A Gelman, J B Carlin, H S Stern, and D B Rubin. (1995) Bayesian Data Analysis. Chapman & Hall, London, UK, herein incorporated by reference). Bayes networks may be constructed as follows:

1. The covariation between each pair of bases bᵢ and bⱼ in the region of interest is evaluated.

This may be done using the mutual information I between positions i and j using the following equation (4):

$$I ( i ; j ) = {\sum }_{{b}_{i}} {\sum }_{{b}_{j}} p ( {b}_{i} , {b}_{j} ) ln ( \frac{p ( {b}_{i} , {b}_{j} )}{p ( {b}_{i} ) p ( {b}_{j} )} ) \qquad ( 4 )$$

Alternatively, if true and pseudo sequence training sets are available, a cross-entropy function may be calculated using the following equation (5):

Hx(i;j)=|Iₜᵣᵤₑ(i;j)−I_{pseudo}(i;j)|

2. A complete graph G is constructed such that each vertex Vᵢ of G corresponds to base position i in the region of interest and each edge connecting vertices Vᵢ and Vⱼ, Eᵢⱼ, is weighted using the values calculated in step 1.

3. The maximum weight spanning tree (MWST) of G may be constructed using an algorithm familiar to those skilled in the art (J Pearl (1991) Probabilistic Reasoning in Intelligent systems: Networks of Plausible Inference, revised 2^{nd} edition. Morgan Kaufmann, San Mateo, Calif., herein incorporated by reference).

4. A root vertex is chosen. This may be the vertex corresponding to the 5′-most base in the region of interest. The base composition of the root position may be calculated using the following equation (6):

$${bnet}_{0 , b} = ln ( \frac{{f}_{pseudo} ( b )}{{f}_{true} ( b )} ) \qquad ( 6 )$$

where fₛₑₜ(b) is defined as the observed frequency of base b at the root position in a given training set.

5. The edges of the MWST are directed away from the root vertex. For each edge in the MWST, the base composition of the destination vertex of the edge, Vⱼ, conditioned on the base composition of the edge's parent vertex, Vᵢ, may be calculated using the following equation (7):

$${bnet}_{b1 , b2} ( i , j ) = ln [ \frac{{p}_{pseudo} ( {b}_{j} = b2 \vert {b}_{i} = b1 )}{{p}_{true} ( {b}_{j} = b2 \vert {b}_{i} = b1 )} ] \qquad ( 7 )$$

where:

b1, b2ε{A, C, G, T}; and

bᵢ, bⱼ=base observed at positions i and j; and

Pₜᵣᵤₑ, P_{pseudo}=probabilities calculated on true and pseudoinitiator codons

Frame-specific monomer composition may be calculated using the following equation (8):

$$f {base}_{b , P} = ln ( \frac{{f}_{b , P , pseudo}}{{f}_{b , P , true}} ) \qquad ( 8 )$$

where f_{b,P,set} is the frequency of base b in the P^{th} codon position in a given training set.

In general, codon usage may be calculated using the following equation (9):

$${codon}_{ t} = ln ( \frac{{f}_{ t , pseudo}}{{f}_{ t , true}} ) \qquad ( 9 )$$

where f_{t,set} is the frequency of triplet t in a given training set.

For analysis of EST sequences, which may be prone to frameshift errors, codon usage in all three coding frames must be tabulated, according to the following equation (10):

$${codon}_{ t , F} = \frac{{f}_{ t , F , pseudo}}{{f}_{ t , F , true}} \qquad ( 10 )$$

where f_{t,F,set} is the frequency of triplet t in coding frame F in a given training set.

Bulk Monomer composition statistics may be computed using the following equation (11):

$${monomer}_{b} = ln ( \frac{{f}_{ b , pseudo}}{{f}_{ b , true}} ) \qquad ( 11 )$$

where f_{b,set} is the frequency of base b in a given training set.

Bulk n-mer composition statistics may be gathered so that coding sequences and untranslated regions may be modeled using Markov chains. Raw n-mer frequencies may be used to root the Markov chains and calculated using the following equation (12):

$${nmer}_{0 , N} = ln ( \frac{{f}_{N , pseudo}}{{f}_{N , true}} ) \qquad ( 12 )$$

where f_{N,set} is the frequency of n-mer N in a given training set.

For each n-mer N consisting of bases b₁b₂ . . . bₙ, conditional n-mer probabilities may be calculated using the following equation (13):

$${nmer}_{N} = ln ( \frac{{p}_{pseudo} ( {b}_{n} \vert {b}_{1} {b}_{2} … {b}_{n - 1} )}{{p}_{true} ( {b}_{n} \vert {b}_{1} {b}_{2} … {b}_{n - 1} )} ) \qquad ( 13 )$$

where pₛₑₜ(bₙ|b₁b₂ . . . bₙ₋₁) is the probability of observing base bₙ following the (n−1)-mer b₁b₂ . . . bₙ₋₁ in a given training set.

Protein coding sequences may generally exhibit a tendency for every third base to be the same. To quantify this effect (known as 3-base periodicity or autocorrelation) the frequency of words ANNA, CNNC, GNNG, and TNNT may be measured, where the N's may be any nucleic base (e.g. A, C, G, or T). These frequencies will depend on base composition. For example, the word TNNT will naturally be found more often in T-rich sequences regardless of coding status. To compensate for this, when measuring autocorrelation the statistical training set is divided into groups (or bins) based on composition.

For the basic version of the autocorrelation score, each sequence may be assigned a bin by the G+C content of its coding sequence. Each bin may be 10% wide, i.e., sequences between 40% and 50% GC may be assigned to a single bin. For each bin, the log probability of autocorrelation in each base may be measured using the following equation (14):

$${auto1}_{B , bin} = ln ( \frac{{n}_{BNNB}}{{n}_{B}} ) \qquad ( 14 )$$

where:

Bε{A, C, G, T}; and

n_{BNNB}=number of BNNB words observed; and

n_{B}=number of bases B observed; and

Training set is the entire coding sequence (excluding the start and stop codons) of each sequence in the statistical training set; and

No pseudo training set is required.

Because the basic autocorrelation score may exhibit an unwanted dependency on the length of its evaluation window, a length-compensated autocorrelation score may be utilized. In the statistical characterization, each sequence may be assigned to 4 different bins based, respectively, on A, C, G, and T content. As before, each bin may be 10% wide. The autocorrelation probabilities in each bin may be calculated using the following equation (15):

$${auto2}_{B , bin} = \frac{{n}_{BNNB}}{{n}_{B}} \qquad ( 15 )$$

where the variables and training set are as defined in equation 14.

The present invention may use one or more scoring functions with one or more parameters to analyze a sequence in the vicinity of an ATG triplet in a nucleic acid sequence and to return a numerical score that quantifies how much said ATG triplet resembles a translation initiation codon.

As used herein, the term “vicinity” refers to nucleic acid sequence surrounding a given location. The terms “upstream vicinity” and “upstream” refer to nucleic acid sequence 5′ to a given location. The terms “downstream vicinity” and “downstream” refer to nucleic acid sequence 3′ to a given location.

The term “scoring function” as used herein refers to a mathematical formula that measures a feature of a nucleic acid sequence. Scoring functions may be used to produce, given any sample window of nucleic acid sequence, a number or vector intended to measure the degree to which a sample sequence resembles an initiator codon. A scoring function may be used for evaluating a set of sequence fragments under a given set of parameters. Scoring functions may be used to generate a feature variable for use in Quadratic Discriminant Analysis. Scoring functions are well known to those skilled in the art (J W Fickett and C-S Tung (1992) “Assessment of protein coding measures.” Nucleic Acids Research 20(24): 6441-6450, herein incorporated by reference).

As used herein, the term “scoring function class” refers to a group of scoring functions. In one embodiment, the scoring functions are selected from those provided in Table 2. Table 2 lists six broad classes of scoring functions and scoring functions comprising each class. Each class, and the individual scoring functions comprising the class, are described herein.

TABLE 2

| Scoring Function Class | Scoring Functions |
|---|---|
| Signal Sequence (SS) | Bayes Network Score |
| Signal Position (SP) | ATG Position Score |
| | log ATG Position Score |
| Bulk Composition (BC) | Bulk Monomer Score |
| | Bulk n-mer Score |
| Transition (T) | Basic Transition Score |
| | Probabilistic Transition Score |
| Coding Composition | Frame-specific Monomer Score |
| (CoC) | Codon Score |
| | Upstream Codon Score |
| | Codon Transition Score |
| Complexity (Comp) | Bulk n-mer Entropy Score |
| | Word Entropy Score |
| | Low Complexity Word Score |
| | Third Base Entropy Score |
| Periodicity (P) | Basic Autocorrelation Score |
| | Length-Compensated Autocorrelation |
| | Score |
| | Fourier Score |
| | Mutual Information Score |

Unless otherwise stated, scores may be calculated as log odds ratios. Scores whose results are named LO_xxx herein may be converted to Bayesian probabilities before use in the quadratic discriminant function.

Signal sequence scoring functions may be used to measure the similarity between a sequence of interest and a known sequence motif, which in this case may be the Kozak consensus. Because of the unique sequence features of the Kozak consensus, a Bayes network may be used to perform this calculation.

The Bayes Network Score may be calculated using the following equation (16):

$$LO\_bnet = {bnet}_{0 , b ( root )} + {\sum }_{i , j pairs} {bnet}_{b ( i ) , b ( j )} ( i , j ) \qquad ( 16 )$$

where:

bnet_{b(i),b(j)}=Bayes network weights as calculated in equation 7; and

bnet_{0,b(root)}=Root weight as calculated in equation 6; and

i, j pairs are the optimal i, j pairs for a given training set as determined in steps 1 through 4 of the Bayes network training procedure described above.

In addition to the nucleic acid sequence of a biological signal, the signal's location within a given sequence is often important to its function. The ribosome-scanning model of translation initiation predicts that initiator codons should be found near the 5′ end of an mRNA.

An ATG Position Score may be used, which is simply the number of bases from the 5′ end of a sequence to a candidate ATG. In addition, since the lengths of biological entities such as exons and untranslated regions may often be lognormally distributed, while Quadratic Discriminant Analysis expects normally distributed data, the log ATG Position Score may also be tested, which is the natural logarithm of the ATG position score.

Protein coding and noncoding sequences generally may differ greatly in their base composition. These differences may sometimes be amplified by breaking a sequence down into small overlapping words. The bulk composition scoring functions may be used to measure these compositional differences.

The Bulk Monomer Score is calculated using the following equation (17):

$$LO\_monomer ( S , begin , end ) = {\sum }_{i = begin}^{end} {monomer}_{{b}_{i}} \qquad ( 17 )$$

where:

S=sequence of bases b₁, b₂, b₃ . . . bₙ; and

Monomer_{b}=monomer composition values as determined using equation 11and appropriate training sets; and

For upstream calculation begin=first base of sequence and end=last base before ATG; and

For downstream calculation begin=first base of ATG and end=50 bases downstream of said ATG.

Three Bulk n-mer Scores may be used in the present invention: the Bulk Dimer, Bulk Trimer, and Bulk Hexamer Scores. These can be calculated using the following equation (18):

$$LO\_nmer ( S , begin , end ) = {nmer}_{0 , {n}_{begin}} + {\sum }_{i = begin + 1}^{end} {nmer}_{{n}_{i}} \qquad ( 18 )$$

where:

S=sequence of overlapping n-mers n₁, n₂, n₃ . . . nₘ; and

nmer_{0,n} values from equation 12; and

nmerₙ values from equation 13; and

for upstream calculation begin=first base of the sequence and end=last base of sequence before the ATG; and

for downstream calculation begin=first base of the ATG and end=50 bases downstream of said ATG.

Transition scores may be sensitive to the presence of a boundary between two statistically distinct regions of a DNA sequence. This boundary in some cases may be the boundary between the 5′ non-coding region and the coding region of a gene, where initiator codons are located. Calculating a transition score may begin by evaluating a scoring function separately on the regions upstream and downstream of a candidate ATG. The Basic Transition Score may be calculated using the following equation (19):

transition_score=downstream_score−upstream_score (19)

If the output of the scoring function involved is a probability, a probabilistic transition score may also be used. The Probabilistic Transition Score may be calculated using the following equation (20):

Pₜᵣₐₙₛ=downstream_score×(1−upstream_score) (20)

where the result is the probability that the candidate ATG is located at a sequence boundary.

Particular transition scores may be named after the scoring function used to evaluate the upstream and downstream regions; for example, the term “codon transition score” refers to a transition score calculated by subtracting an upstream codon score from a downstream codon score.

In addition to the general compositional differences between coding and noncoding sequence, the first, second, and third bases of individual codons exhibit distinct compositional biases. Coding composition-scoring functions make use of this phenomenon.

Coding composition-based scoring functions are sensitive to frameshift errors in their data. Because such errors may occur frequently in sequence data, the coding composition scoring functions employ a frameshift compensation strategy. This involves computing an estimate of the probability of some frame F being the true coding frame at a distance d from the start codon using the following equations (21):

p_{F}(d)=(1−s)p_{F}(d−1)+(s/2)[p_{F-1}(d−1)+p_{F+1}(d−1)]

p₀(d=0)=1;

p₋₁(0)=p₊₁(0)=0

where s is the estimated frameshift rate. Generally s=0.001 is used. Use of these frame probabilities in the scoring functions is discussed below.

Before use in the Frame-Specific Monomer Scoring Functions (e.g. First Base Score, Second Base Score, and Third Base Score), the composition statistics calculated in equation 8 may be frameshift compensated using the following equation (22):

$${fs\_fbase}_{b , d , π} = ln \{ {\sum }_{F = - 1 , 0 , + 1} exp [ {fbase}_{b , π + F} {p}_{F} ( d ) ] \} \qquad ( 22 )$$

where:

b={A, C, G, T}; and

d=distance from initiator codon; and

π=codon position of interest; and

fbase=frame-specific monomer usage values from equation 8; and

p_{F}(d)=frame probabilities as calculated in equation 21.

The frameshift compensated usage statistics may then be used to calculate a log odds frame-specific monomer composition score for a given sequence using the following equation (23):

$$LO\_fmono ( S , begin , end ) = {\sum }_{i = begin}^{end} {fs\_fbase}_{{t}_{i , π} , \vert i - begin \vert , π} \qquad ( 23 )$$

where:

S=sequence of nonoverlapping triplets t₁, t₂, t₃ . . . tₙ; and

t_{i,π}=base π of triplet tᵢ; and

π=codon position of interest.

Codon usage statistics may be frameshift-compensated for the Codon score in a manner similar to that described above using the following equation (24):

$${fs\_codon}_{t , d} = ln [ {\sum }_{F = - 1 , 0 , + 1} {codon}_{t , F} {p}_{F} ( d ) ] \qquad ( 24 )$$

where:

tε{AAA, AAC, AAG . . . }; and

d=distance from initiator codon; and

codon=codon usage values from equation 10; and

p_{F}(d)=frame probabilities as calculated in equation 21.

These codon usage statistics are then used to calculate a log odds ratio score using the following equation (25):

$$LO\_codon ( S , begin , end ) = {\sum }_{i = begin}^{end} {fs\_codon}_{{t}_{i} , \vert i - begin \vert} \qquad ( 25 )$$

where the sequence S is broken down into nonoverlapping triplets t₁, t₂, t₃ . . . tₙ.

Protein-coding sequences may often appear to be comprised of nearly random sequence, while noncoding sequences may appear to be comprised of long stretches of one- and two-base repeats. Coding sequence may thus be said to be more complex than noncoding sequence. The complexity of a sequence may be measured using the Shannon's entropy equation (26):

$$H = - {\sum }_{s} {f}_{s} l g {f}_{s} \qquad ( 26 )$$

where fₛ is the frequency of symbol s in a sequence, and lg represents the logarithm base 2.

Given a region of a nucleic acid sequence between positions start and stop, the Bulk n-mer Entropy Score may be measured as the Shannon entropy calculated on the n-mer frequencies of the entire fragment (for example see C E Shannon (1948) “A Mathematical Theory of Communication” The Bell System Technical Journal 27: 379-423 and 623-656 incorporated herein by reference). To calculate the Word Entropy Score, the region between start and stop may be divided into overlapping words and the Shannon entropy may be calculated for each word. In one embodiment of the present invention, this may be done using overlapping 8-base words. The word entropy score of the region may then be the average of these individual word entropies. The Low Complexity Word Frequency Score performs the same word wise entropy calculation as the Word Entropy Score, but instead measures the frequency of words scoring less than a given cutoff.

The Third Base Entropy Score may be measured based on Shannon's joint entropy function equation (27):

$$H ( x , y ) = - {\sum }_{i , j} {f}_{x = i , y = j} l g {f}_{x = i , y = j} \qquad ( 27 )$$

where

x and y are two different positions in a sequence; and

i,jε{A, C, G, T}; and

f_{x=i, y=j} is the frequency at which the base at y is j when x is i.

High joint entropy is observed between bases x and x+3 of coding sequences; therefore, the Third Base Entropy Score is H(x, x+3).

As stated above, coding sequences may tend to repeat every third base. The periodicity scores below may be used to quantify this tendency.

To calculate the Basic Autocorrelation Score, a sequence fragment may be first assigned to a bin based on G+C content as described above. Sequences with G+C content lower than the lowest bin may be assigned to the lowest bin; likewise for sequences whose G+C content is higher than the highest bin. The autocorrelation score may then be calculated using the following equation (28):

$$LO\_autocorr1 = {\sum }_{B} {n}_{BNNB} ( ln \frac{{n}_{B}}{{n}_{tot}} - {auto1}_{B , bin} ) \qquad ( 28 )$$

where:

Bε{A, C, G, T}; and

n_{B}=number of bases B counted; and

n_{BNNB}=number of BNNB autocorrelations counted; and

nₜₒₜ=total bases in the fragment; and

auto1_{B,bin}=log probabilities from equation 14.

For the Length-Compensated Autocorrelation Score (the length-compensated version of the autocorrelation score), sequences may be binned in a manner similar to that used for the basic autocorrelation score, except that 4 different bins may be assigned based on A, C, G, and T content. The score may be calculated. using the following equation (29):

$$\begin{matrix}LO\_autocorr2 = {\sum }_{B} {n}_{BNNB} ln ( \frac{{n}_{B} / {n}_{tot}}{{auto2}_{B , bin ( B )}} ) + \\ ( {n}_{B} - {n}_{BNNB} ) ln ( \frac{1 - {n}_{B} / {n}_{tot}}{1 - {auto2}_{B , bin ( B )}} )\end{matrix} \qquad ( 29 )$$

where:

Bε{A, C, G, T}; and

n_{B}=number of bases B counted; and

n_{BNNB}=number of BNNB autocorrelations counted; and

nₜₒₜ=total bases in the fragment; and

auto2_{B,bin(B)}=log probabilities from equation 15.

To describe the Fourier Score, the function EQ on a sequence of bases s₁, s₂, s₃ . . . may be defined using the following equation (30):

$$EQ ( p , q ) = \{ \begin{matrix}1 \begin{matrix}if {s}_{p} = {s}_{q} & \end{matrix} & & \\ 0 \begin{matrix}if {s}_{p} \neq {s}_{q} & \end{matrix} & & \end{matrix} \qquad ( 30 )$$

The Fourier Score for a sequence fragment between positions start and stop may be the third Fourier coefficient of the EQ function. This may be calculated using the following equation (31):

$$fourier = {\sum }_{p = start}^{stop} {\sum }_{q = p}^{stop} EQ ( p , q ) {ⅇ}^{2 π ( q - p ) i / 3} \qquad ( 31 )$$

The Mutual Information Score may be used to measure periodic relationships between bases other than the strict identities that: may be measured by the Autocorrelation and Fourier Scores. The mutual information between bases separated by a periodic distance (p) may be defined using the following equation (32):

MI(p)=H(x)+H(x+p)−H(x,x=p)

where the functions H are the simple and joint Shannon entropy functions of equation 26 and equation 27.

A scoring function may be applied with one or more parameter sets to one or more locations in a nucleic acid sequence in a training set to produce feature variables for testing in Quadratic Discriminant Analysis. The present invention uses Quadratic Discriminant Analysis with two or more feature variables to generate a quadratic discriminant function. In one embodiment, the feature variables are selected from the list of feature variables provided as Table 3. Table 3 column headings: (a) the “Feature Variable Name” column lists the unique identifier given to each feature variable, (b) the “Feature Variable Class” column lists the class to which each feature variable belongs as defined by the scoring function used to generate said feature variable, (c) the “Location” column provides the beginning and end of the region of the sequence scored by said feature variable, either as (1) absolute base position for begin and end; (2) position relative to the candidate ATG (denoted as ATG+n or ATG−n, where n is the relative offset in bases); or as (3) transition, in which a score for the region upstream of an ATG is subtracted from its corresponding downstream region score, (d) the “Prior” column provides the prior (π) for Bayesian probability-based scoring function for said feature variable, and (e) the “Other Information” column provides values of other miscellaneous parameters for said feature variable.

TABLE 3

| Feature | Feature Variable | Location | | | Other |
|---|---|---|---|---|---|
| Variable Name | Class | Begin | End | Prior | Information |
| net | Bayes network | ATG − 9 | ATG + 5 | 0.5 | |
| net1 | Bayes network | ATG − 9 | ATG + 5 | 0.1 | |
| net8 | Bayes network | ATG − 9 | ATG + 5 | 0.8 | |
| ulcn | ATG position | 1 | ATG − 1 | | |
| llcn | Log ATG position | 1 | ATG − 1 | | |
| umon | Bulk monomer | 1 | ATG − 1 | 0.5 | |
| umon1 | Bulk monomer | 1 | ATG − 1 | 0.1 | |
| umon8 | Bulk monomer | 1 | ATG − 1 | 0.8 | |
| dmon | Bulk monomer | ATG + 0 | ATG + 50 | 0.5 | |
| dmon1 | Bulk monomer | ATG + 0 | ATG + 50 | 0.1 | |
| dmon8 | Bulk monomer | ATG + 0 | ATG + 50 | 0.8 | |
| tmon | Bulk monomer | transition | | 0.5 | |
| tmon1 | Bulk monomer | transition | | 0.1 | |
| tmon8 | Bulk monomer | transition | | 0.8 | |
| up2 | Bulk n-mer | 1 | ATG − 1 | 0.5 | n = 2 |
| up21 | Bulk n-mer | 1 | ATG − 1 | 0.1 | n = 2 |
| up28 | Bulk n-mer | 1 | ATG − 1 | 0.8 | n = 2 |
| dn2 | Bulk n-mer | ATG + 0 | ATG + 50 | 0.5 | n = 2 |
| dn21 | Bulk n-mer | ATG + 0 | ATG + 50 | 0.1 | n = 2 |
| dn28 | Bulk n-mer | ATG + 0 | ATG + 50 | 0.8 | n = 2 |
| tr2 | Bulk n-mer | transition | | 0.5 | n = 2 |
| tr21 | Bulk n-mer | transition | | 0.1 | n = 2 |
| tr28 | Bulk n-mer | transition | | 0.8 | n = 2 |
| up3 | Bulk n-mer | 1 | ATG − 1 | 0.5 | n = 3 |
| up31 | Bulk n-mer | 1 | ATG − 1 | 0.1 | n = 3 |
| up38 | Bulk n-mer | 1 | ATG − 1 | 0.8 | n = 3 |
| dn3 | Bulk n-mer | ATG + 0 | ATG + 50 | 0.5 | n = 3 |
| dn31 | Bulk n-mer | ATG + 0 | ATG + 50 | 0.1 | n = 3 |
| dn38 | Bulk n-mer | ATG + 0 | ATG + 50 | 0.8 | n = 3 |
| tr3 | Bulk n-mer | transition | | 0.5 | n = 3 |
| tr31 | Bulk n-mer | transition | | 0.1 | n = 3 |
| tr38 | Bulk n-mer | transition | | 0.8 | n = 3 |
| ufm0 | Frame-specific monomer | 1 | ATG − 1 | 0.5 | frame = 0 |
| ufm01 | Frame-specific monomer | 1 | ATG − 1 | 0.1 | frame = 0 |
| ufm08 | Frame-speeific monomer | 1 | ATG − 1 | 0.8 | frame = 0 |
| dfm0 | Frame-specific monomer | ATG + 0 | ATG + 50 | 0.5 | frame = 0 |
| dfm01 | Frame-specific monomer | ATG + 0 | ATG + 50 | 0.1 | frame = 0 |
| dfm08 | Frame-specific monomer | ATG + 0 | ATG + 50 | 0.8 | frame = 0 |
| tfm0 | Frame-specific monomer | transition | | 0.5 | frame = 0 |
| tfm01 | Frame-specific monomer | transition | | 0.1 | frame = 0 |
| tfm08 | Frame-specific monomer | transition | | 0.8 | frame = 0 |
| ufm1 | Frame-specific monomer | 1 | ATG − 1 | 0.5 | frame = 1 |
| ufm11 | Frame-specific monomer | 1 | ATG − 1 | 0.1 | frame = 1 |
| ufm18 | Frame-specific monomer | 1 | ATG − 1 | 0.8 | frame = 1 |
| dfm1 | Frame-specific monomer | ATG + 0 | ATG + 50 | 0.5 | frame = 1 |
| dfm11 | Frame-specific monomer | ATG + 0 | ATG + 50 | 0.1 | frame = 1 |
| dfm18 | Frame-specific monomer | ATG + 0 | ATG + 50 | 0.8 | frame = 1 |
| tfm1 | Frame-specific monomer | transition | | 0.5 | frame = 1 |
| tfm11 | Frame-specific monomer | transition | | 0.1 | frame = 1 |
| tfm18 | Frame-specific monomer | transition | | 0.8 | frame = 1 |
| ufm2 | Frame-specific monomer | 1 | ATG − 1 | 0.5 | frame = 2 |
| ufm21 | Frame-specific monomer | 1 | ATG − 1 | 0.1 | frame = 2 |
| ufm28 | Frame-specific monomer | 1 | ATG − 1 | 0.8 | frame = 2 |
| dfm2 | Frame-specific monomer | ATG + 0 | ATG + 50 | 0.5 | frame = 2 |
| dfm21 | Frame-specific monomer | ATG + 0 | ATG + 50 | 0.1 | frame = 2 |
| dfm28 | Frame-specific monomer | ATG + 0 | ATG + 50 | 0.8 | frame = 2 |

| | | | | |
|---|---|---|---|---|
| tfm2 | Frame-specific monomer | transition | 0.5 | frame = 2 |
| tfm21 | Frame-specific monomer | transition | 0.1 | frame = 2 |
| tfm28 | Frame-specific monomer | transition | 0.8 | frame = 2 |

| | | | | |
|---|---|---|---|---|
| ufsc | Codon | 1 | ATG − 1 | 0.5 |
| ufsc1 | Codon | 1 | ATG − 1 | 0.1 |
| ufsc8 | Codon | 1 | ATG − 1 | 0.8 |
| dfsc | Codon | ATG + 3 | ATG + 50 | 0.5 |
| dfsc1 | Codon | ATG + 3 | ATG + 50 | 0.1 |
| dfsc8 | Codon | ATG + 3 | ATG + 50 | 0.8 |

| | | | |
|---|---|---|---|
| tfsc | Codon | transition | 0.5 |
| tfsc1 | Codon | transition | 0.1 |
| tfsc8 | Codon | transition | 0.8 |

| | | | | |
|---|---|---|---|---|
| hux | Bulk entropy | 1 | ATG − 1 | n = 1 |
| hdx | Bulk entropy | ATG + 0 | ATG + 50 | n = 1 |

| | | | |
|---|---|---|---|
| thx | Bulk entropy | transition | n = 1 |

| | | | |
|---|---|---|---|
| hu8 | Word entropy | 1 | ATG − 1 |
| hd8 | Word entropy | ATG + 0 | ATG + 50 |

| | | |
|---|---|---|
| th8 | Word entropy | transition |

| | | | | |
|---|---|---|---|---|
| ulc10 | Low complexity word frequency | 1 | ATG − 1 | cutoff = 1.0 |
| ulc15 | Low complexity word frequency | 1 | ATG − 1 | cutoff = 1.5 |
| dlc10 | Low complexity word frequency | ATG + 0 | ATG + 50 | cutoff = 1.0 |
| dlc15 | Low complexity word frequency | ATG + 0 | ATG + 50 | cutoff = 1.5 |

| | | | |
|---|---|---|---|
| tlc10 | Low complexity word frequency | transition | cutoff = 1.0 |
| tlc15 | Low complexity word frequency | transition | cutoff = 1.5 |

| uhxy | Third base entropy | 1 | ATG − 1 | |
|---|---|---|---|---|
| dhxy | Third base entropy | ATG + 0 | ATG + 50 | |
| thxy | Third base entropy | transition | | |
| uac | Basic autocorrelation | 1 | ATG − 1 | 0.5 |
| uac1 | Basic autocorrelation | 1 | ATG − 1 | 0.1 |
| uac8 | Basic autocorrelation | 1 | ATG − 1 | 0.8 |
| dac | Basic autocorrelation | ATG + 0 | ATG + 50 | 0.5 |
| dac1 | Basic autocorrelation | ATG + 0 | ATG + 50 | 0.1 |
| dac8 | Basic autocorrelation | ATG + 0 | ATG + 50 | 0.8 |
| tac | Basic autocorrelation | transition | | 0.5 |
| tac1 | Basic autocorrelation | transition | | 0.1 |
| tac8 | Basic autocorrelation | transition | | 0.8 |
| uak | Length-compensated autocorrelation | 1 | ATG − 1 | 0.5 |
| uak1 | Length-compensated autocorrelation | 1 | ATG − 1 | 0.1 |
| uak8 | Length-compensated autocorrelation | 1 | ATG − 1 | 0.8 |
| dak | Length-compensated autocorrelation | ATG + 0 | ATG + 50 | 0.5 |
| dak1 | Length-compensated autocorrelation | ATG + 0 | ATG + 50 | 0.1 |
| dak8 | Length-compensated autocorrelation | ATG + 0 | ATG + 50 | 0.8 |

| | | | |
|---|---|---|---|
| tak | Length-compensated autocorrelation | transition | 0.5 |
| tak1 | Length-compensated autocorrelation | transition | 0.1 |
| tak8 | Length-compensated autocorrelation | transition | 0.8 |

| | | | |
|---|---|---|---|
| uft3 | Fourier | 1 | ATG − 1 |
| dft3 | Fourier | ATG + 0 | ATG + 50 |

| | | |
|---|---|---|
| tft3 | Fourier | transition |

| | | | | |
|---|---|---|---|---|
| umi1 | Mutual information | 1 | ATG − 1 | period = 1 |
| umi2 | Mutual information | 1 | ATG − 1 | period = 2 |
| umi3 | Mutual information | 1 | ATG − 1 | period = 3 |
| dmi1 | Mutual information | ATG + 0 | ATG + 50 | period = 1 |
| dmi2 | Mutual information | ATG + 0 | ATG + 50 | period = 2 |
| dmi3 | Mutual information | ATG + 0 | ATG + 50 | period = 3 |

| | | | |
|---|---|---|---|
| tmi1 | Mutual information | transition | period = 1 |
| tmi2 | Mutual information | transition | period = 2 |
| tmi3 | Mutual information | transition | period = 3 |

The present invention uses any combination of feature variables comprising a minimum of one variable from, each of any two variable classes. This combination is used to generate a quadratic discriminant function using Quadratic Discriminant Analysis. In one embodiment the correlation coefficient for a variable combination should be greater than 0.8. In another embodiment, the correlation coefficient for a variable combination should be greater than 0.9.

The term “feature variable” as used herein refers to a variable whose value quantifies a particular characteristic of a nucleic acid sequence. The term “variable” as used herein refers to a quantity that may assume any one of a set of values.

As used herein, the term “variable class” refers to a group of variables comprised of variables derived from a common scoring function.

The term “quadratic discriminant function” as used herein refers to a mathematical formula that can be used to classify objects based on a set of feature variable values. The optimal parameters for a quadratic discriminant function to use when applied to a particular problem can be discovered through Quadratic Discriminant Analysis.

The terms “Quadratic Discriminant Analysis” and “QDA” refer to a statistical multivariate classification method well known to those skilled in the art.

As used herein, the term “variable selection” refers to a process of selecting a set of variables for use in a quadratic discriminant function. Variable selection may be carried out using a cross validation procedure.

As used herein, the term “cross validation” refers to a process of evaluating different sets of variables. Cross validation sequence sets may be created by randomly dividing a, discriminant: training data set into a QDA training set (75% of the sequences) and a QDA testing set (25% of the sequences). Multiple validation sets may be randomly generated in this manner. For a given combination of feature variables, a, quadratic discriminant function may be trained on each QDA training set and then tested on the corresponding QDA test set. The average performance on the training sets may be used to rate how well that quadratic discriminant function performs.

How well a quadratic discriminant function performs may be measured using a correlation coefficient (CC). As used herein, the terms “correlation coefficient” and “CC” refer to a numerical value calculated using the following equation (34):

$$CC = \frac{TP \cdot TN - FP \cdot FN}{\sqrt{( TP + FP ) ( TP + FN ) ( TN + FP ) ( TN + FN )}} \qquad ( 34 )$$

where:

TP=number of true positives; and

TN=number of true negatives; and

FP=number of false positives; and

FN=number of false negatives; and

To average the performance over all of the cross-validation sets, the total number of TP, TN, FP, and FN reported in all 10 cross validation sets may be used in equation 33. A quadratic discriminant function that produces a correct classification in 100% of cases may have a CC=1. Random guessing may yield on average a CC=0. A quadratic discriminant function that produces an incorrect classification in 100% of cases may have a CC=−1.

Variables may be selected using a greedy algorithm, which is a process well known to those skilled in the art. A greedy algorithm attempts to progressively maximize a quantity by making the largest possible increase at each step. Starting with a, single variable as the basis, all two-variable quadratic discriminant functions that utilize the basis plus one of the other variables may be trained and tested. The best two-variable combination may be selected as the new basis, and three-variable QDFs may then be trained and tested in a similar manner. Eventually a point may be reached where adding more variables does not improve performance significantly and the process may be stopped.

The greedy algorithm may not produce the best combination of variables on the first try. Therefore, a refining process may also be used, wherein a combination produced by the greedy algorithm may be altered by removing variables and/or replacing one variable with other variables not already in the combination. Each of these new variable combinations may be cross validated. If a higher-scoring combination is found, that combination may be used as a starting point for a new round of greedy algorithm selection.

Maximizing quadratic discriminant function performance (in terms of CC) is one aspect of the present invention. Minimizing the number of variables involved in the quadratic discriminant function is another aspect of the present invention. It is well known to one skilled in the art that performance may be improved by adding more variables to a quadratic discriminant function, but this may also make the function dependent on features not common to all of the training data A function in fewer variables, on the other hand, may be more robust, i.e. less prone to errors caused by peculiarities in its training set. Therefore, variable combinations may be selected that yield the best performance for the number of variables involved.

Once a variable combination is selected it may be used to generate a quadratic discriminant function that is trained for use in a computer program. This quadratic discriminant function may then be trained on the entire training set.

The quadratic discriminant function generates a probability score for ATG triplets in a nucleic acid sequence based on the output: of a combination of two or more of the scoring functions described herein. The probability score is used to classify ATG triplets as initiator codons or as pseudoinitiator codons.

The present invention uses the quadratic discriminant function to analyze an unknown ATG in a nucleic acid sequence. The ATG and its surrounding sequence may first be evaluated using variables selected through the variable selection process described above. Based on these scores, the quadratic discriminant function may be used to determine how closely this set of scores resembles the true initiator model and the pseudoinitiator model. A Bayesian model selection process may be used to calculate the probability that the candidate ATG is an initiator codon; if this value is above a user-defined threshold, the ATG maybe reported as a true initiator codon.

As used herein, the term “user-defined threshold” refers to a minimum desirable score defined by the user.

### EXAMPLES

### Example 1

Program for detecting initiator codons in an EST sequence data set

To build the statistical and discriminant data sets for detecting initiator codons in a Zea mays EST sequence data, 894 Zea mays cDNA sequences reported to contain a protein coding sequence were obtained from GenBank. Redundant sequences were removed, resulting in a set of 442 unique cDNA sequences.

These 442 cDNA sequences were compared with EST sequences in a second database.

This produced three types of sequences:

I. No match: 167 cDNA sequences that had no matching EST sequence; and

II. Partial match: 119 cDNA sequences that matched one or more EST sequences, but for whom the matching region of the EST sequence did not cover the start codon; and

III. Complete match: 156 cDNA sequences that matched EST sequences for whom the matching region of the EST sequence contained the reported initiator codon.

These sequence groups were then used to create the following training sets:

1) Coding sequence statistical training set: To create this set, the first group of 167 cDNA sequences (I) was used. This set is provided as the “mrna_no_hit.fa” file.

2) Positive Bayes network training set: To create this set, the first and second groups of cDNA sequences (I) and (II) were combined. From this combined group, (a) partial sequences (no start codon), (b) incorrectly annotated sequences, (c) sequences with the start codon <10 nucleotides from the beginning of the sequence, and (d) sequences containing ambiguous base symbols near the initiator codon were removed. This resulted in 157 sequences whose initiator codons were used as the positive Bayes network training set that is provided as the “true_sites.fa” file.

3) Negative Bayes network training set: Pseudoinitiators from the first group of 167 cDNA sequences (I) were collected. From these, (a) pseudoinitiators <10 nucleotides from the beginning of the sequence, and (b) sequences containing ambiguous base symbols near the initiator codon were discarded. The resulting 2163 pseudoinitiators comprise the negative Bayes network training set that is provided as the “pseu_sites.fa” file.

4) Positive discriminant training set: This set contains the initiator codon regions of the 156 EST sequences in the third group (III). This set is provided as the “true.fa” file. The header line of each sequence contains the annotation “/start=(number)” which indicates the location of the true initiator codon.

5) Negative discriminant training set: The non initiator ATG triplets (pseudoinitiators) found in the 156 EST sequences in the third group (III) comprise the negative discriminant training set that is provided as the “pseu.fa” file. The header line of each sequence contains the annotation “/start=(number)”, which indicates the ATG in the sequence that was used.

The coding sequence statistical training set and the positive and negative Bayes network training sets were used for the statistical characterization of the initiator and pseudoinitiator codons. The parameters produced from the statistical characterization of this training set are contained in the “param_files.tar.gz” files which contains the following files:

(a) init.net: (Kozak consensus); and

(b) f_mono.score (frame-specific base composition); and

(c) codon.odds (codon usage); and

(d) monomer.score (monomer composition); and

(e) 2mers.local.score (bulk dimer composition); and

(f) 3mers.local.score (bulk trimer composition); and

(g) autocorr.dat (basic autocorrelation); and

(h) auto3.dat (length-compensated autocorrelation).

The positive and negative Bayes network training sets were used to generate the init.net file (a) above; all other files listed above were produced by analyzing the coding sequence statistical training set.

Scoring functions were applied to the positive and negative discriminant training sets to generate values for the feature variables. Cross validation sets were established and the variable selection process described above was carried out using the program referred to herein as “xval3”, the source code of which is included as the “xval3.tar.gz” files. Xval3 accepted as input a list of cross validation sets to train and test upon and a list of feature variable combinations to try. Xval3 outputed the correlation coefficient of each combination tested. This output is provided in Table 4. Table 4 contains a listing of the variable combinations tested along with their measured correlation coefficient and the number of variables in the combination from each scoring function class. The greedy algorithmic addition of variables and the refinement process were carried out through the repeated use of xval3 on potentially useful variable combinations.

The following variable combination was then selected for training the quadratic discriminant function for use in the program developed for detecting initiator codons in Zea mays sequence data, referred to herein as the Codon1 program and provided as the “source.tar.gz” files.

| CC | Variable Combination | SS | SP | CoC | BC | P | Com |
|---|---|---|---|---|---|---|---|
| 0.902 | net8 ulen dfm2 tfsc tmon1 | 1 | 1 | 2 | 1 | 0 | 0 |

Final training of the QDF was performed using the −f option of xval3. In this mode, xval3 trained a discriminant on all of the data provided—no test set was reserved—and wrote the resulting quadratic discriminant function parameters to the “init.qdf” file. The Zea mays-trained program was used to find the initiator codon for a data set containing Zea mays cDNA sequences. The test sequences are provided as SEQ ID NOS: 1-5. The codon1 output format for each sequence is:

>sequence name

position [frame] POS/neg score

position [frame] POS/neg score

where each line below a named sequence represents an ATG codon found in that sequence. The “position” field is provided as a number and represents the position of the first base of the ATG in question relative to the 5′ end in the sequence. The “frame” field is the position modulo 3. Modulo refers to the remainder left after dividing one number into another. For instance, 5 modulo 3=2 (5/3=1 remainder 2). This technique is used to assign identifiers to the 3 reading frames (referred to as 0, 1, and 2) of a DNA sequence where 1=reading frame 1; 2=reading frame 2; 3=reading frame 0; 4=reading frame 1; 5=reading frame 2; 6=reading frame 0; 7=reading frame 1; etc. This is useful when there are two positive predictions in a sequence, so that you can compare and see if they′ re in the same reading frame. The “POS/neg” field indicates whether codon1 is calling that ATG an initiator codon (POS) or a non-initiator codon. The “score” field provides the score assigned by the QDF to the ATG in question.

SEQ ID NO 1

CGCTGATCCCACCGGCTGATAGAGTTGGCGGCCGGGGGAGTGAGTCAGGG
ATGGCGTCGGAGCCGGTGGCGCGGGCGGTGGCGGAGGAGGTGGGCCGCTG
GGGCAGCATGAAGCAGACGGGGGTGACCCTGCGGTACATGATGGAGTTCG
GCTCCCGCCCCACCCAGCGAAACCTGCTCCTCTCCGCGCAGTTCCTGCAC
AAGGAGCTCCCCATCCGCTTCGCACGCCGCGCGCTCGAGCTCGACTCGCT
GCCCTTCGGCCTCTCCAACAAGCCCGCCATCCTCAAGGTGCGGGACTGGT
ACTTGGACTCATTCCGGGACATCAGATACTTCCCTGAAGTGAGGAGCCGG
AACGACGAGCTCGCTTTCACGCAGATGATCAATATGGTCAAGGTGCGGCA
TAACAATGTGGTTCCAACCATGGCCTTGGGAGTGCAGCAGCTGAAGAAGG
AGCTGGGCCGCTCAAGGAAGGTTCCATTCGAAGTCGATGAGATCGACGAG
TTCCTTGACCGGTTCTACATGTCAAGGAATGGCATTCGCATGCTGATAGG
GCAGCATGTGGCTTTGCATGACCCTAAACCGGAG

SEQ ID NO 1

51 [0] POS 0.996263
108 [0] neg 0.000154769
138 [0] neg 4.62666e−14
141 [0] neg 4.33354e−11
375 [0] neg 6.40591e−113
384 [0] neg 3.72451e−120
406 [1] neg 1.55892e−133
420 [0] neg 6.41363e−136
487 [1] neg 2.12325e−211
519 [0] neg 8.35899e−241
529 [1] neg 1.53068e−253

SEQ ID NO 2

GAGCTTTCCCGTGAGGAAAATGTGTACATGGCGAAGCTCGCTGAGCAGGC
GGAGAGGTACGAGGAGATGGTTGAGTTCATGGAGAAGGTAGCCAAGACTG
TTGACTCGGAGGAGCTCACTGTGGAGGAGCGAAACCTCTTGTCTGTTGCA
TACAAGAACGTCATTGGAGCCCGCCGCGCCTCATGGCGCATCATCTCCTC
CATCGAGCAGAAGGAGGAGGGCCGAGGCAATGAGGACCGAGTAACACTCA
TCAAGGATTATCGTGGCAAGATTGAGACTGAGCTGACCAAGATCTGTGAT
GGCATCCTCAAGCTGCTTGAGACCCATCTTGTGCCGTCTTCCACTGCCCC
CGAGTCCAAGGTCTTCTATCTCAAGATGAAGGGTGATTACTACAGATACC
TTGCTGAGTTCAAGACTGGAGCTGAGAGAAAGGACGCCGCTGAGAACACG
ATGGTGGCATACAAGGCTGCCCAAGACATTGCTCTGGCTGAGCTTGCTCA
ACTTACCTTTTAAGGTTGGACTGGCACTTAACTTCTTAGTGTGCTACTAT
GAGATTCTGAACTAACCT

SEQ ID NO 2

```
  20 [2] neg 0.000168116
  28 [1] POS 0.949223
  67 [1] neg 0.00576387
  79 [1] neg 0.00203849
183 [0] neg 3.01406e−25
230 [2] neg 1.00698e−41
299 [2] neg 7.10241e−75
376 [1] neg 9.2949e−119
451 [1] neg 2.59959e−174
```

SEQ ID NO 3

TATCTACTATACTATACTCTAGGAAGCAAGGACACCACCGCCATGGCAGC
CAAGATGCTTGCATTGTTCGCTCTCCTAGCTCTTTGTGCAAGCGCCACTA
GTGCGACCCATATTCCAGGGCACTTGCCACCAGTCATGCCATTGGGTACC
ATGAACCCATGCATGCAGTACTGCATGATGCAACAGGGGCTTGCCAGCTT
GATGGCGTGTCCGTCCCTGATGCTGCAGCAACTGTTGGCCTTACCGCTTC
AGACGATGCCAGTGATGATGCCACAGATGATGACGCCTAACATGATGTCA
CCATTGATGATGCCGAGCATGATGTCACCAATGGTCTTGCCGAGCATGAT
GTCGCAAATGATGATGCCACAATGTCACTGCGACGCCGTCTCGCAGATTA
TGCT

SEQ ID NO 3

```
  43 [1] POS 0.50187
  55 [1] neg 0.148864
136 [1] neg 1.31051e−11
151 [1] neg 1.30474e−14
159 [0] neg 5.43224e−17
163 [1] neg 5.67366e−16
175 [1] neg 3.99587e−24
178 [1] neg 1.39405e−23
202 [1] neg 1.80511e−27
220 [1] neg 7.21893e−34
256 [1] neg 1.15482e−52
265 [1] neg 4.0763e−58
268 [1] neg 2.65293e−56
277 [1] neg 3.52697e−60
280 [1] neg 6.05344e−64
292 [1] neg 1.78791e−70
295 [1] neg 1.55481e−71
307 [1] neg 1.47441e−81
310 [1] neg 3.29559e−77
319 [1] neg 4.32753e−85
322 [1] neg 4.21433e−89
331 [1] neg 1.82254e−95
346 [1] neg 1.14871e−104
349 [1] neg 1.31313e−103
```

SEQ ID NO 4

CTGTTCACAAATTATATGCAATCGCAAGCGAGCAGAATGGCGAGGTCCAG
TGGTAGTAGACCAGTGGCCCTCGTGCTGCTGGCGCTGTGCGCCGCCGCCC
TCTCGTCGGCCACGGTGACCGTGAATGAGCCCATCGCCAATGGCCTCTCC
TGGAGCTTCTACGACGTTTCCTGCCCGTCGGTGGAGGGCATCGTGCGCTG
GCACGTCGCCGAGGCCCTCCGCCGCGACATCGGCATCGCCGCGGAGCTCA
TCCGCATCTTCTTCCACGACTGCTTCCCGCATGGCTGCGACGCGTCCGTC
CTCCTGTCTGGTTCCATCAGCGAGCAGATCGTAGTACCCAACCAGACGC

SEQ ID NO 4

```
  16 [1] neg 1.2232e−05
  37 [1] POS 0.990987
125 [2] neg 5.33434e−13
140 [2] neg 1.0461e−12
281 [2] neg 6.83312e−56
```

SEQ ID NO 5

CCCACGCGTCCTCGCGACTGGCATTATTCATGTGGAACAAATCACTACAA
AGTGCTGATGGATAAGTTTCACCTCGTATCCACTGCCTTCCTGGAGCTTG
GTCAAGGCTATCAAAAGGCAATCGAAGAAATCACTAGGCGAATGGGAGCA
GGAATGGCAAAATTTATATGCAAGGAGGTTGAAACTGTTGATGACTATGA
CGAGTATTGTCACTATGTAGCCGGGCTAGTTGGTTATGGACTTTCCAGGC
TCTTTTATGCTGCTGGGACGGAAGATCTGGCTCCAGATTCACTATCAAAT
TCAATGGGTCTCTTTTTACAGAAAACTAATATAATTAGGGATTATTTGGA
GGACATAAATGAGATACCAAAGTCCCGCATGTTCTGGCCTCGAGAGATAT
GGAGTAAATATGCAGATAAACTCGAGGATTTCAAATATGAGGAAAATACC
GAAAAGGCAGTACAATGCTTGAACGATATAGTGACGAATGCACTGATTCA
TGCTG

SEQ ID NO 5

```
  30 [0] neg 2.6951e−05
  58 [1] neg 0.0202753
142 [1] neg 4.71748e−19
154 [1] neg 1.3063e−15
168 [0] neg 2.00425e−26
191 [2] neg 1.8025e−31
197 [2] neg 7.25371e−34
215 [2] neg 4.72543e−41
236 [2] neg 1.25242e−44
257 [2] neg 4.82022e−58
304 [1] neg 3.79128e−82
359 [2] neg 8.85961e−120
379 [1] neg 3.10287e−128
399 [0] neg 8.58869e−143
410 [2] neg 8.13857e−152
437 [2] neg 1.47469e−173
```

The computer program files provided herein (*.c.ascii) and (*.h.ascii) contain source code listings for codon1. These files can be used directly to build the codon1 program if the “.ascii” extension is stripped off of the filenames prior to compilation using a standard C++compiler.

1) A method for finding translation initiation codons in a nucleotide sequence, comprising:
a) analyzing a first data, set to measure a combination of features of initiator codons and pseudoinitiator codons and to produce a set of numerical values for said combination of features; and
b) evaluating scoring functions by reading a sequence in the vicinity of an ATG triplet and using said scoring functions and said scoring function's parameters to return a numerical score that quantifies how much said ATG triplet resembles an initiator codon; and
c) generating a quadratic discriminant function through selection of a combination of feature variables that optimally classifies ATG triplets in a nucleotide sequence as initiator codons or as pseudoinitiator codons based on the output of said scoring functions and through the use of Quadratic Discriminant Analysis; and
d) using said quadratic discriminant function to analyze a second data set of nucleotide sequences by evaluating at least one scoring function for each ATG triplet in said sequences and to calculate the probability of an initiator codon at a position using the output of said analysis.

2) A method for finding translation initiation codons in a nucleotide sequence, as recited in claim 1, wherein said combination of features from step a) comprises at least two of the features provided in Table 1.

3) A method for finding translation initiation codons in a nucleotide sequence, as recited in claim 1, wherein said scoring functions from step d) comprise at least two of the scoring functions provided in Table 2.

4) A method for finding translation initiation codons in a nucleotide sequence, as recited in claim 1, wherein said combination of feature variables from step c) comprises any combination of at least two of the feature variables provided in Table 3 wherein the combination comprises one feature variable from each of any two feature variable classes and results in a correlation coefficient for the feature variable combination of greater than 0.9.

5) A method for finding translation initiation codons in a nucleotide sequence, as recited in claim 1, wherein said combination of feature variables from step c) comprises any combination of at least two of the feature variables provided in Table 3 wherein the combination comprises one feature variable from each of any two feature variable classes and results in a correlation coefficient for the feature variable combination of greater than 0.8.
